(19) United States
(12) Patent Application Publication
(10) Pub. No.: US 2024/0424176 A1
(43) Pub. Date: Dec. 26, 2024

(54) BREAST PUMP WITH MILK FLOW SENSOR

(71) Applicant: Dr. Brown's Company, St. Louis, MO (US)

(72) Inventor: Douglas HANNEKEN, St. Louis, MO (US)

(21) Appl. No.: 18/705,250

(22) PCT Filed: Oct. 6, 2022
(86) PCT No.: PCT/US2022/077655

§ 371 Date: Apr. 26, 2024

Related U.S. Application Data
(60) Provisional application No. 63/263,271, filed on Oct. 29, 2021.

(51) Int. Cl. A61M 1/06

(52) U.S. Cl. CPC A61M 1/064; A61M 2205/3389; A61M 2205/502

(57) ABSTRACT

A breast pump includes a cup assembly for engaging at least a portion of a breast including a nipple and an area surrounding the nipple, a vacuum pump assembly for applying a vacuum to the cup assembly, a container fluidly connected to the cup assembly for receiving milk expressed from the nipple of the breast, a sensor assembly positioned in the container, and a controller. The sensor assembly includes a first contact and a second contact. The controller includes a processor and a memory. The memory includes instructions that when executed by the processor configure the controller to periodically detect a measurement indicative of a capacitance between the first and second contacts of the sensor assembly, and operate the vacuum pump assembly to apply a vacuum to the cup assembly based at least in part on the detected measurement indicative of the capacitance between the first and second contacts.

### CROSS REFERENCE TO RELATED APPLICATION

This application claims the benefit of priority to U.S. Provisional Patent Application No. 63/263,271, filed Oct. 29, 2021, the contents of which are incorporated by reference herein in their entirety.

### BACKGROUND

The field of this disclosure relates generally to breast pumps and more particularly to a breast pump that includes a sensor for monitoring the flow of milk during operation.

Breast pumps, whether electric or manually operated, typically include at least one breast cup configured for sealing placement over a nursing mother's breast. A vacuum pump is operatively connected to the breast cup for applying a vacuum to the mother's breast within the cup. More specifically, commonly configured breast cups have a central passage for receiving at least the mother's nipple and more typically some adjacent portion of the mother's breast, allowing vacuum pressure to be applied to the mother's nipple for extracting milk. During use, the vacuum pressure is often applied in pulses, with the central passage being sometimes vented between pulses. A bottle or other suitable container is usually in fluid connection with the breast cup to collect the extracted milk.

When a baby is placed at the breast to be fed, a cascade of events occurs. The baby places their mouth and tongue with a negative pressure of approximately 30 mm Hg (millimeters of mercury) to the nipple/areola (latches) and stimulates milk ejection through a series of quick, shallow sucks referred to as non-nutritive suckling. Non-nutritive suckling consists of stable lengths of sucking bursts and durations of pauses. The average pressure of non-nutritive suckling is approximately 70 to 90 mm Hg.

As the baby performs non-nutritive suckling, the mother's brain recognizes the stimulation at the breast and a reflex arc occurs. This reflex arc causes an oxytocin release from the posterior pituitary, which ultimately leads to milk ejection. Oxytocin is a hormone that acts on the myoepithelial cells eliciting a contraction of the smooth muscle cells around the alveolus in the breast. The contraction of these cells actively pushes the milk into the ducts toward the nipple, where the milk is ejected. The baby acts as a milk collector by means of nutritive suckling with strong, even draws. During nutritive suckling, the movement of the tongue, jaw, and swallowing facilitates milk flow. The average pressure for nutritive suckling is approximately 75-100 mm Hg.

Vacuum pressure needed to extract milk using a conventional breast pump is substantially higher than that of a suckling infant. For example, the vacuum pressure applied to the mother's breast by conventional breasts pump is often 200 mm Hg and greater. Over the full transfer period, such high vacuum pressure can often be painful to the mother and, in some cases, can irritate or damage the mother's breast tissue. Moreover, applying pulses of vacuum pressure to the mother's breast does not adequately simulate the peristaltic movements of an infant's mouth and tongue during breastfeeding to apply oral pressure to the mother's breast. Furthermore, at least some known breast pumps are configured such that the mother cannot see the milk collecting in the container, thereby making it difficult for the mother to determine if milk is flowing or when the container is full.

### SUMMARY

In one aspect, a breast pump includes a cup assembly for engaging at least a portion of a breast including a nipple and an area surrounding the nipple, a vacuum pump assembly for applying a vacuum to the cup assembly, a container fluidly connected to the cup assembly for receiving milk expressed from the nipple of the breast, a sensor assembly positioned in the container, and a controller. The sensor assembly includes a first contact and a second contact. The controller includes a processor and a memory. The memory includes instructions that when executed by the processor configure the controller to periodically detect a measurement indicative of a capacitance between the first contact and the second contact of the sensor assembly, and operate the vacuum pump assembly to apply a vacuum to the cup assembly based at least in part on the detected measurement indicative of the capacitance between the first contact and the second contact of the sensor assembly.

In another aspect, a method of operating a breast pump includes periodically detecting a measurement indicative of a capacitance between a first contact and second contact of a sensor assembly positioned in a container for receiving milk expressed from a nipple of a breast, and operating a vacuum pump assembly to apply a vacuum to a cup assembly for engaging at least a portion of the breast including the nipple and an area surrounding the nipple based at least in part on the detected measurement indicative of the capacitance between the first contact and second contact of the sensor assembly.

### BRIEF DESCRIPTION OF THE DRAWINGS

FIG. 1 is a front perspective of one embodiment of a wearable electric breast pump.

FIG. 2 is a rear perspective of the breast pump of FIG. 1.

FIG. 3 is a simplified schematic diagram of the breast pump of FIG. 1.

FIG. 4 is a front perspective of a container and sensor assembly of the breast pump of FIG. 1.

FIG. 5 is a side view of the container and sensor assembly of FIG. 4 with the container shown transparent.

FIG. 6 is a front perspective of the sensor assembly of the breast pump of FIG. 1

FIG. 7 is a rear perspective of the sensor assembly of FIG. 6.

FIG. 8 is a rear view of the sensor assembly of FIG. 6.

FIG. 9 is a front view of the sensor assembly of FIG. 6.

FIG. 10 is a side view of the sensor assembly of FIG. 6.

FIG. 11 is a cross-section of the sensor assembly of FIG. 6 viewed similar to the side view of FIG. 10.

### DETAILED DESCRIPTION OF THE DRAWINGS

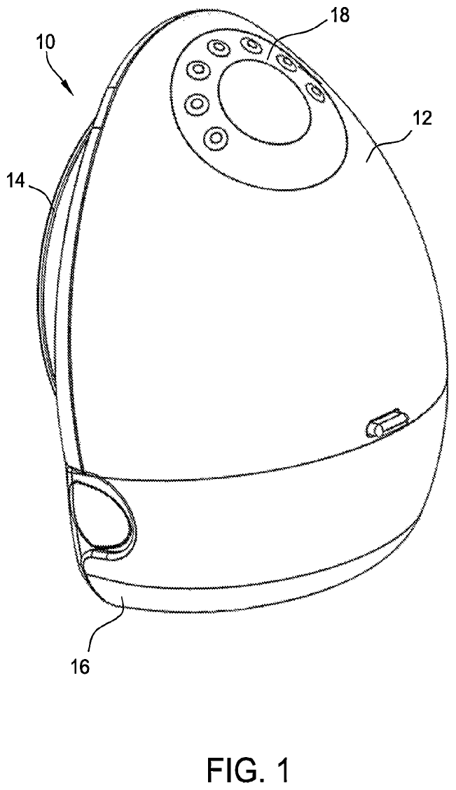

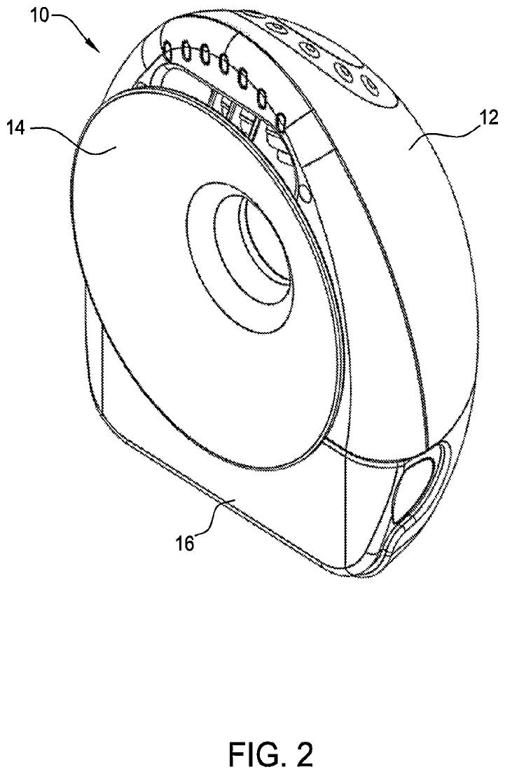

With reference now to the accompanying drawings, and specifically to FIGS. 1 and 2, a wearable electric breast pump according to one suitable embodiment is illustrated and is indicated generally at 10. Although aspects of this disclosure are described with reference to a wearable electric breast pump, it should be understood that the disclosure is not so limited, and aspects of this disclosure may be used with any suitable type of breast pump including both electric and manual breast pumps.

As seen in FIGS. 1 and 2, the breast pump 10 includes a suitable housing, indicated generally at 12, for housing various working components such as a pump or pumps, a controller, and other components as will be described later herein. The breast pump 10 also comprises a cup assembly 14 (best illustrated in FIG. 2) for receiving a mother's breast, and a container 16 for receiving milk expressed from the nursing mother's breast. The cup assembly 14 is in fluid communication with the container 16, such as via a fluid path, such as ducting, passages, tubing, or the like (not shown).

A user interface 18 is positioned on the housing 12 of the breast pump 10. The user interface 18 includes a display for displaying information to the user and one or more buttons for receiving input from the user. Some embodiments do not include the user interface on the housing 12, but instead may communicate with a remote device (such as a mobile phone, tablet, desktop computer, laptop computer, or the like) and utilize the remote device as a user interface.

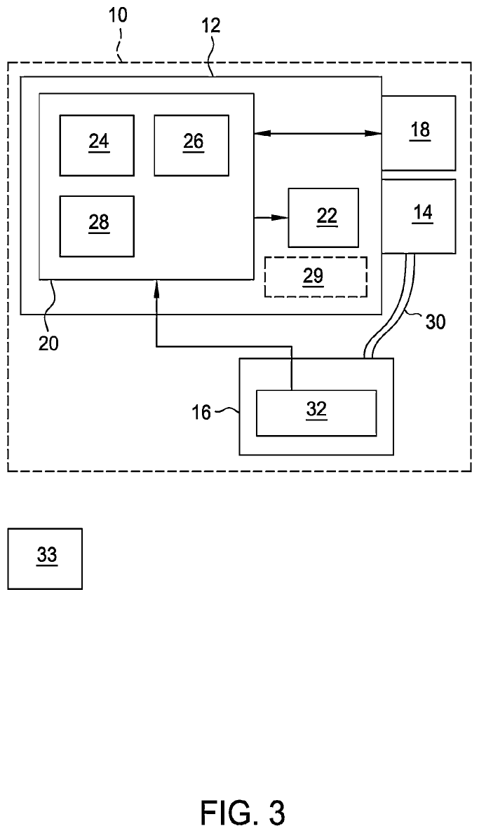

FIG. 3 is a simplified schematic diagram of the breast pump 10 of FIG. 1. The housing 12 of the breast pump 10 encloses a controller 20 and a vacuum pump assembly 22. The controller 20 includes a processor 24, a memory 26, and a communications interface 28. The vacuum pump assembly 22 includes at least one vacuum pump, and may include other components to aid in controlling the vacuum pressure produced by the vacuum pump. Moreover, the vacuum pump assembly 22 may include one or more positive pressure pumps to apply positive pressure to portions of the cup assembly 14 to mimic more closely a baby suckling at the mother's breast. The breast pump 10 optionally includes a tilt sensor 29 to detect an orientation of the breast pump (e.g., level or some angle off of level in the x-y plane of the breast pump).

In general, instructions stored in the memory 26, when executed by the processor 24, configure the controller 20 to control operation of the breast pump 10. The controller 20 is communicatively coupled to the vacuum pump assembly 22 and controls operation of the vacuum pump assembly 22 according to the instructions stored in the memory 26 and according to user input through the user interface 18. In some embodiments, the breast pump 10 includes more than one vacuum pump assembly 22 controlled by the controller 20. The cup assembly 14 is attached to the housing 12 of the breast pump and is in communication with the vacuum pump assembly 22 to allow the vacuum pump assembly 22 to apply a vacuum to the cup assembly 14.

The cup assembly 14 is in fluid communication with the container 16 through a fluid path 30. The user interface 18 is attached to the housing 12 and communicatively coupled to the controller 20. It should be understood that the user interface 18 and the cup assembly 14 may be at least partially housed within the housing 12. A sensor assembly 32 is positioned in the container 16 and communicatively coupled to the controller 20.

As used herein, a “processor” may be one or more central processing units, microprocessors, microcontrollers, reduced instruction set circuits (RISC), application specific integrated circuits (ASIC), logic circuits, and any other circuit or processor capable of executing the functions described herein. A “memory” may include, but is not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above are examples only and are thus not intended to limit in any way the definition and/or meaning of the terms “processor” or memory. In some embodiments, the processor 24 and the memory 26 are separate components, while in other embodiments, the processor 24 and the memory 26 are part of a single component, such as a microcontroller. In the example embodiment, the communications interface 28 is a wireless communications module. In other embodiments, the communications interface 28 may be any suitable wired or wireless communications module.

In the example embodiment, the communications interface 28 comprises a Bluetooth® adapters. In other embodiments, the communications interface 28 may include one or more of a radio frequency (RF) transceiver, a Bluetooth® adapter, a Wi-Fi transceiver, a ZigBee® transceiver, an infrared (IR) transceiver, and/or any other device and communication protocol for wireless communication. (Bluetooth is a registered trademark of Bluetooth Special Interest Group of Kirkland, Washington: ZigBee is a registered trademark of the ZigBee Alliance of San Ramon, California.) In embodiments using wired communication interfaces, any suitable wired communication protocol for direct communication may be used, including, without limitation, USB, RS232, I2C, SPI, analog, and/or proprietary I/O protocols. In some embodiments, the wired communication interface includes a wired network adapter allowing the controller 20 to be coupled to a network, such as the Internet, a local area network (LAN), a wide area network (WAN), a mesh network, and/or any other network to communicate with remote devices and systems via the network.

The communications interface 28 allows the controller 20 to communicate with a user's remote device 33. In the example embodiments, the remote device 33 is a mobile phone or a tablet computer. In some embodiments, the remote device 33 is a laptop computer, a desktop computer, a personal digital assistant (PDA), or any other device operable to receive data, display data, and/or receive user input.

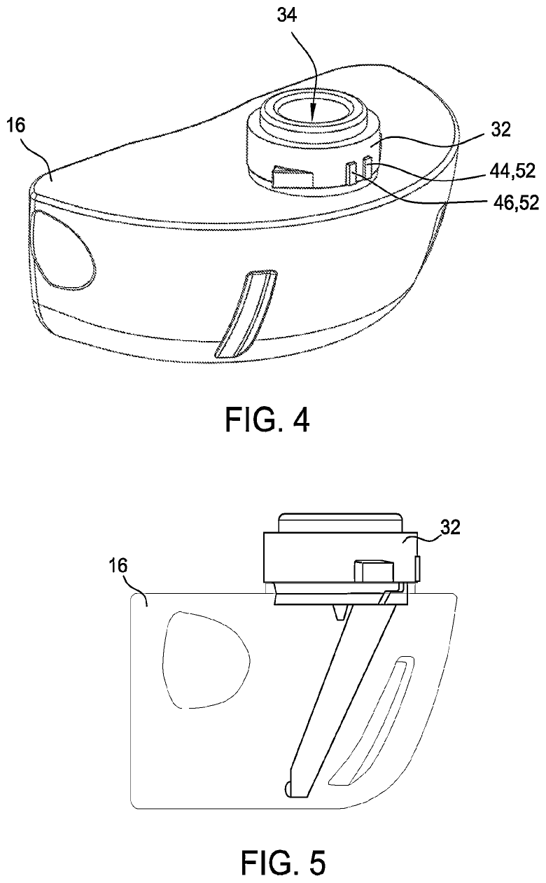

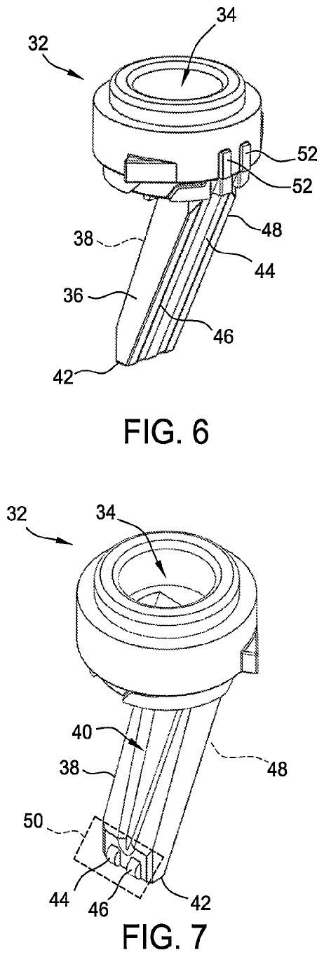

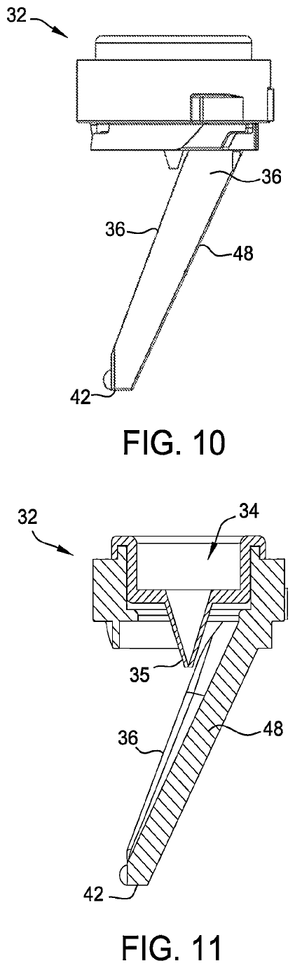

FIGS. 4 and 5 are perspective views of the container 16 removed from the housing 12. Suitably, the container 16 can be selectively engaged and disengaged from the housing 12 by the user. In FIGS. 4 and 5, the sensor assembly 32 is positioned in the container 16. In FIGS. 6-11, the sensor assembly is removed from the container 16.

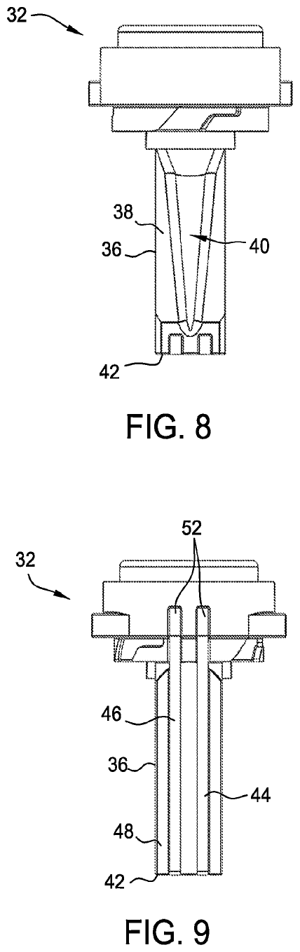

The sensor assembly 32 includes an opening 34 through which expressed milk passes from the fluid path 30 into the container 16. The sensor assembly 32 includes a ramp 36 positioned beneath the opening 34 and extending towards a bottom of the container 16 (when the sensor assembly is positioned within the container 16). When the breast pump 10 is in use, drops of milk fall through the opening 34, through a duck bill valve 35 (FIG. 11) onto a first side 38 of the ramp 36, and down a channel 40 (FIG. 8) in the first side of the ramp toward a bottom 42 of the ramp (and toward the bottom of the container 16). In other embodiments, the ramp 36 does not include the channel 40.

The angle of the ramp 36 relative to the container 16 facilitates milk hitting the ramp within an expected range of tilting of the breast pump (and therefore the attached container) during use, while minimizing the volume occupied by the ramp, which is volume that would otherwise be able to hold milk. In the illustrated embodiment, the ramp 36 extends substantially to the bottom of the container 16. In some embodiments, the ramp 36 touches the bottom of the container 16 when the sensor assembly is positioned in the container 16, while in other embodiments, there is a small gap between the bottom of the ramp 36 and the bottom of the container 16.

The sensor assembly 32 also includes a first contact 44 and a second contact 46 positioned on the ramp 36. The first and second contacts 44, 46 are generally rectangular contacts extending down a second side 48 of the ramp 36 opposite the first side 38 of the ramp 36. The first and second contacts 44, 46 are electrically conductive contacts. In the example embodiment, the contacts 44, 46 are stainless steel contacts. Other embodiments may use contacts made from any suitable conductive material. The first and second contacts 44, 46 wrap around the bottom 42 of the ramp 36 and a portion of each of the first and second contacts 44, 46 extends to a lower portion 50 (FIG. 7) of the first side 38 of the ramp. An upper portion 52 of each of the contacts 44, 46 extends to a position that is located outside of the container 16 (when installed in the container 16) to make electrical connection to the controller 20 through corresponding contacts and electrical connections (not shown) in the housing 12.

The first and second contacts 44, 46 operate as a variable capacitor, whose capacitance varies based on the material between the first and second contacts 44, 46. When the container 16 is empty, atmospheric air is the dielectric of the capacitor formed by the first and second contacts 44 and 46, and a certain initial capacitance will exist between the first and second contacts 44, 46. When the container 16 is filled with milk (which has a different relative permeability than air), the milk will be between the first and second contacts 44, 46, thereby changing the capacitance. In between an empty container and a full container, portions of the first and second contacts 44, 46 will have air between them and portions will have milk between them, resulting in different capacitances than when the container is empty or full. Thus, by monitoring the capacitance and changes to the capacitance between the first contact 44 and the second contact 46, the controller 20 can determine if the container 16 contains any milk, when milk starts to enter the container 16, the rate at which milk is entering the container 16, and the level (or volume) of milk in the container 16.

In general, during operation of the breast pump 10, the controller 20 is configured, by the instructions stored in the memory 26 and executed by the processor 24, to periodically detect a measurement indicative of a capacitance between the first and second contacts 44, 46 of the sensor assembly 32, and to operate the vacuum pump assembly 22 to apply a vacuum to the cup assembly 14 based at least in part on the detected measurement indicative of the capacitance between the first and second contacts 44,46. The capacitance between the first contact 44 and the second contact 46 may be measured using any suitable techniques for measuring capacitance. For example, with a known resistance connected to the first and second contacts 44, 46 to form an RC circuit, a step voltage may be applied and the RC time constant (the time taken to reach approximately 63.2% of the applied voltage) of the RC circuit may be measured. The measurement indicative of the capacitance between the first and second contacts 44, 46 can be the RC time constant, or the actual capacitance may be calculated from the RC time constant and the known resistance, and the determined capacitance may be used as the measurement indicative of the capacitance between the first and second contacts 44, 46. Any other measured or calculated value that varies in a known way with the capacitance between the first and second contacts 44, 46 may be used as the measurement indicative of the capacitance between the first and second contacts 44, 46.

When a mother starts operation of the breast pump 10 with an empty container 16, the controller determines an initial value of the detected measurement indicative of the capacitance between the first and second contacts 44, 46. Either while determining the initial value or after determining the initial value, the controller 20 begins to operate the vacuum pump assembly 22 to cyclically apply and relax vacuum pressure to the cup assembly 14 and thereby to at least the nipple of the breast. At this initial stage, the controller 20 operates the vacuum pump assembly 22 in a first operating mode. In the example embodiment, the first operating mode is a stimulating mode of pumping cycle of the breast pump. The stimulating mode is designed to mimic an infant's initial suckling (e.g., non-nutritive suckling), which causes the mother to experience “letdown.” “Letdown” occurs when milk within the mother's breast flows toward the nipple.

In the first operating (e.g., stimulating) mode, the pump 22 is operated to apply a suction (e.g., maximum) vacuum pressure to the mother's breast. For example, a maximum vacuum pressure in the range of about 30 mm Hg to about 150 mm Hg, more suitably in the range of about 75 mm Hg to about 125 mm Hg, is applied to the breast. In one particularly suitable embodiment, the suction vacuum pressure is applied to the mother's breast continuously throughout the cycle. It is understood, however, that the suction vacuum pressure can be selectively varied throughout the cycle. The controller 20 continues to operate the vacuum pump assembly 22 in the first operating mode until the mother experiences letdown.

The controller 20 detects the occurrence of letdown by continuing to periodically detect the measurement indicative of the capacitance between the first and second contacts 44, 46. Prior to letdown, no milk will flow into the container and no milk will contact the first and second contacts 44, 46. Thus, the capacitance will remain substantially the same. Once letdown occurs and milk start to flow, the milk passes through the opening 34 and is directed onto the first side 38 of the ramp 36, down the channel 40, and onto the portion of the contacts 44, 46 at the bottom 42 of the ramp 36. The presence of milk will cause the capacitance between the first and second contacts 44, 46 to fluctuate, and therefore the measurement indicative of the capacitance between the first and second contacts 44, 46 will fluctuate.

When the controller 20 detects a change in the measurement indicative of the capacitance between the first and second contacts 44, 46, the controller 20 determines that letdown has occurred and the controller 20 then operates the vacuum pump assembly in a second operating mode. In some embodiments, the controller 20 does not determine that letdown has occurred until the measurement indicative of the capacitance between the first and second contacts 44, 46 varies from the initial value by more than a threshold amount in order to avoid a noisy signal or other erroneous measurement causing a premature switch to the second operating mode. In some embodiments, the controller 20 does not determine that letdown has occurred until the measurement indicative of the capacitance between the first and second contacts 44, 46 varies from the initial value in more than one periodic measurement.

The second operating mode is an expressing mode. The expressing mode of the pumping cycle is suitably designed to simulate the suckling action and frequency of a nursing infant, e.g., the peristaltic movement of the infant's tongue and palate used to express milk. In particular, during each cycle the vacuum pump assembly 22 is operated to apply a suction (e.g., maximum) vacuum pressure to the mother's breast. For example, a vacuum pressure in the range of about 70 mm Hg to about 150 mm Hg, and more suitably in the range of about 75 mm Hg to about 125 mm Hg, is applied. In one embodiment, the suction vacuum pressure is applied to the mother's breast in the range of about 50 to about 80 percent of each cycle, and more suitably of about 70 percent of each cycle.

As the controller 20 continues to operate the vacuum pump assembly 22 in the second operating mode, the controller 20 also continues to periodically detect the measurement indicative of the capacitance between the first and second contacts 44, 46 to determine the amount of milk in the container 16. As milk fills the container, more of the first and second contacts 44, 46 are covered by the milk, and the capacitance between the first and second contacts 44, 46 continues to change. The capacitance depends on how much of the first and second contacts 44, 46 has milk between them, and thereby can be correlated to the height of the milk in the container 16. Because the volume and dimensions of the container 16 is known and fixed, the controller 20 can determine the volume of milk in the container 16 from the detection of the measurement indicative of the capacitance between the first and second contacts 44, 46. This may be done through a look-up table stored in the memory 26 that correlates the measurements to volume of milk, or through an equation that computes the volume of milk from the detection of the measurement indicative of the capacitance between the first and second contacts 44, 46. The determination of the amount of milk in the container 16 is not limited to the volume of milk, and some embodiments may determine a percentage that the container 16 is filled, a height of liquid in the container 16, or any other suitable measurement of the amount of milk.

The correlations between the measurement indicative of the capacitance between the first and second contacts 44, 46 and the amount of milk in the container 16 assume that the container 16 is level. Thus, the determination would be inaccurate if the container 16 was not level. In embodiments that include the tilt sensor 29, the controller 20 checks the orientation of the breast pump 10 (and thereby the attached container 16) using the tilt sensor 29 and corrects the determination of the amount of milk in the container 16 based on the tilt detected by the tilt sensor 29. The controller 20 may correct the values using one or more lookup tables stored in the memory 26 that correlate the measurement indicative of the capacitance between the first and second contacts 44, 46 and the amount of milk for a particular tilt, or may calculate the amount of milk as described above and then apply a correction factor determined from the amount of tilt. The correction factor may be retrieved from memory 26 or may be calculated by the controller 20. In some embodiments, the amount of milk is calculated directly by the controller 20 using an equation that takes the measurement indicative of the capacitance between the first and second contacts 44, 46, the volume and/or dimensions of the container 16, and the tilt into account.

In some embodiments, the controller 20 saves the determined amount of milk during each periodic determination in the memory 26. Using two or more of these determinations, the controller 20 may calculate a rate of flow of the milk. This rate of flow may be used by the controller 20 to tailor the second operating mode to the particular user (such as to attempt to increase the flow if the flow rate is low, or to increase the comfort by decreasing the vacuum pressure if the rate is relatively high), to estimate the remaining time needed to fill the container 16, and/or to inform the user about the rate of flow of milk.

When the controller 20 determines that the amount of milk in the container 16 has reached a threshold value, the controller 20 stops the pumping session by stopping operation of the vacuum pump assembly 22. The threshold value is a value that is substantially the maximum recommended volume of the container 16 (which may be less than the volume of the container 16 in order to avoid spilling or overfilling).

The controller 20 may also output information to the nursing mother using the breast pump 10. The information may be displayed on the user interface 18, and or transmitted using the communications interface 28 to the mobile device 33 for display on the mobile device. The information output to the nursing mother may include a notification of which operating mode is being used, a notification of the detection of letdown, the amount of milk in the container 16, the rate of flow of milk into the container 16, the estimated amount of time remaining until the container 16 is full, a notification that the container 16 is full, and/or any other suitable information.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

What is claimed is:

1. A breast pump comprising:
a cup assembly for engaging at least a portion of a breast including a nipple and an area surrounding the nipple;
a vacuum pump assembly for applying a vacuum to the cup assembly and thereby to at least the nipple of the breast;
a container fluidly connected to the cup assembly for receiving milk expressed from the nipple of the breast;
a sensor assembly positioned in the container, the sensor assembly including a first contact and a second contact; and
a controller including a processor and a memory, the memory containing instructions that when executed by the processor configure the controller to:
periodically detect a measurement indicative of a capacitance between the first contact and the second contact of the sensor assembly; and
operate the vacuum pump assembly to apply the vacuum to the cup assembly based at least in part on the detected measurement indicative of the capacitance between the first contact and the second contact of the sensor assembly.

2. The breast pump as set forth in claim 1, wherein the detected measurement has an initial value at a start of operation of the breast pump and the controller is further configured to operate the vacuum pump assembly in a first operating mode based at least in part on the initial value of the detected measurement.

3. The breast pump as set forth in claim 2, wherein the controller is further configured to operate the vacuum pump assembly in a second operating mode when the detected measurement varies from the initial value, wherein the second operating mode differs from the first operating mode.

4. The breast pump as set forth in claim 3, wherein the controller is further configured to operate the vacuum pump assembly at the second operating mode when the detected measurement varies from the initial value by more than a threshold value.

5. The breast pump as set forth in claim 4, wherein the controller is further configured to operate the vacuum pump assembly at the second operating mode when the detected measurement varies from the initial value by more than the threshold value for more than one of the detected measurement.

6. The breast pump as set forth in claim 1, wherein the controller is further configured to determine, based at least in part on the detected measurement, a volume of milk in the container when the controller is operating the vacuum pump assembly.

7. The breast pump as set forth in claim 6, wherein the controller is configured to determine the volume of milk in the container using a lookup table stored in the memory that correlates the detected measurement to a volume of milk.

8. The breast pump as set forth in claim 6, wherein the controller is configured to determine the volume of milk in the container by calculating the volume of milk in the container using an equation that correlates the detected measurement to a volume of milk.

9. The breast pump as set forth in claim 6, wherein the controller is further configured to stop operating the vacuum pump assembly when the determined volume of milk in the container exceeds a volume threshold value.

10. The breast pump as set forth in claim 6, wherein the controller is further configured to transmit the determined volume of milk in the container to a remote device for display to a user of the breast pump.

11. The breast pump as set forth in claim 1, wherein the controller is further configured to store in the memory each detected measurement that was periodically detected and determine a rate of flow of milk into the container based on two or more of the detected measurement stored in the memory.

12. The breast pump as set forth in claim 11, wherein the controller is further configured to transmit the determined rate of flow of milk to a remote device for display to a user of the breast pump.

13. The breast pump as set forth in claim 1 wherein the sensor assembly comprises a ramp extending toward a bottom of the container, wherein the ramp is positioned to receive the milk expressed from the nipple of the breast and direct the received milk along a first side of the ramp toward the bottom of the container.

14. The breast pump as set forth in claim 13, wherein the first contact and the second contact are positioned on the ramp.

15. The breast pump as set forth in claim 14, wherein the first contact and the second contact are positioned on a second side of the ramp opposite the first side of the ramp.

16. The breast pump as set forth in claim 15, wherein a lower portion of the first contact and the second contact extends to a lower portion of the first side of the ramp adjacent to the bottom of the container.

17. The breast pump as set forth in claim 15, wherein an upper portion of the first contact and the second contact extends to a position outside of the container.

18. A method of operating a breast pump comprising:
periodically detecting a measurement indicative of a capacitance between a first contact and a second contact of a sensor assembly positioned in a container for receiving milk expressed from a nipple of a breast; and
operating a vacuum pump assembly to apply a vacuum to a cup assembly for engaging at least a portion of the breast including the nipple and an area surrounding the nipple based at least in part on the detected measurement indicative of the capacitance between the first contact and second contact of the sensor assembly.

19. The method as set forth in claim 18, wherein the detected measurement has an initial value at the start of operation of the breast pump and operating the vacuum pump assembly comprises operating the vacuum pump assembly in a first operating mode based at least in part on the initial value of the detected measurement.

20. The method as set forth in claim 19, wherein operating the vacuum pump assembly comprises operating the vacuum pump assembly in a second operating mode when the detected measurement varies from the initial value, wherein the second operating mode differs from the first operating mode.

21. The method as set forth in claim 18, further comprising determining, based at least in part on the detected measurement, a volume of milk in the container.

22. The method as set forth in claim 21, wherein operating the vacuum pump assembly comprises stopping operation of the vacuum pump assembly when the determined volume of milk in the container exceeds a volume threshold value.

23. The method as set forth in claim 18, further comprising storing each of the detected measurement that was periodically detected and determining a rate of flow of milk into the container based on two or more of the detected measurement that is stored.
